(19) United States
(12) Patent Application Publication
(10) Pub. No.: US 2025/0387399 A1
(43) Pub. Date: Dec. 25, 2025

(54) TREATMENT OF A SELECTIVE POPULATION OF PATIENTS HAVING DEMENTIA WITH LEWY BODIES

(71) Applicant: EIP Pharma, LLC, Boston, MA (US)

(72) Inventor: John Jahangir Alam, Boston, MA (US)

(73) Assignee: EIP Pharma, LLC, Boston, MA (US)

(21) Appl. No.: 19/312,779

(22) Filed: Aug. 28, 2025

Related U.S. Application Data
(63) Continuation of application No. 18/707,318, filed on May 3, 2024.
(60) Provisional application No. 63/276,529, filed on Nov. 5, 2021.

(51) Int. Cl. A61K 31/519; A61P 25/14; A61P 25/28

(52) U.S. Cl. CPC A61K 31/519; A61P 25/14; A61P 25/28

(57) ABSTRACT

The present invention provides methods and compositions for the treatment of subjects that have Dementia with Lewy Bodies (DLB) but have no substantial tau pathology. Subjects with DLB that have no substantial tau pathology are shown to be particularly responsive to treatment with a selective p38α mitogen activated protein kinase (MAPK) inhibitor compared to subjects with DLB that have substantial tau pathology.

### REFERENCE TO RELATED APPLICATIONS

This application is a continuation under 35 U.S.C. § 120 of U.S. application Ser. No. 18/707,318, filed May 3, 2024, which is a national stage filing under 35 U.S.C. 371 of International Patent Application Serial No. PCT/US2022/049052, filed Nov. 4, 2022, which claims priority under 35 U.S.C. § 119 (e) to U.S. Provisional Patent Application No. 63/276,529, filed Nov. 5, 2022, each of which are hereby incorporated by reference in their entireties.

### BACKGROUND

Dementia with Lewy bodies (DLB) is the second most common dementia after Alzheimer's disease and yet there are no approved therapies to address this progressive disorder.

### SUMMARY

The present disclosure encompasses the discovery that administration of a selective p38 α MAPK inhibitor, neflamapimod, is effective to treatment a select patient population that has Dementia with Lewy Bodies (DLB) but no substantial tau pathology. Specifically, neflamapimod was effective to treat DLB symptoms in patients with no substantial tau pathology characterized by biomarkers of tau pathology, such as levels of phosphorylated tau (ptau) in plasma. Elevated ptau level in plasma is a surrogate measure of tau pathology in brain, which is often associated with Alzheimer's disease pathology. It has been discovered herein that a subset of DLB patients have tau pathology, while another subset of DLB patients do not have substantial tau pathology, and that patients without substantial tau pathology respond differently to neflamapimod therapy. Provided herein are methods of treatment comprising administering neflamapimod to DLB patients that do not have substantial tau pathology.

In some embodiments, provided is a method of treating Dementia with Lewy Bodies (DLB) in a subject having DLB but no substantial tau pathology, the method comprising administering a selective p38α mitogen activated protein kinase (MAPK) inhibitor to the subject.

In some embodiments, the p38α mitogen activated protein kinase (MAPK) inhibitor is neflamapimod.

In some embodiments no substantial tau pathology in a subject is characterized by level of ptau181 in plasma. In some embodiments no substantial tau pathology in a subject is characterized by level of ptau217 in plasma. In some embodiments, no substantial tau pathology in a subject is characterized by positron emission topography (PET) of brain.

In some embodiments, no substantial tau pathology in a subject is associated with a level of amyloid beta (Aβ) 42 in cerebrospinal fluid in the subject that is lower than that of subjects diagnosed as having Alzheimer's Disease.

In some embodiments, no substantial tau pathology in a subject is characterized by a level of plasma ptau lower than that of a subject having Alzheimer's Disease or Alzheimer's Disease related pathology as measured in a Simoa ptau181 assay, or equivalent thereof of another assay methodology that measures plasma ptau.

In some embodiments, no substantial tau pathology in a subject is characterized by a ptau181 level of less than 2.2 pg/mL in plasma. In some embodiments, no substantial tau pathology in a subject is characterized by a ptau181 level of less than 2.2 pg/mL in plasma assessed by Simoa platform assay, or equivalent thereof of another assay methodology.

In some embodiments, provided herein are methods of treating alpha-synuclein associated degenerative disease in a subject having DLB but no substantial tau pathology the methods comprising administering to the subject a selective p38α mitogen activated protein kinase (MAPK) inhibitor.

In some embodiments, provided herein are methods of inhibiting neuronal loss in the central nervous system in a subject having DLB but no substantial tau pathology, the methods comprising administering to the subject a selective p38α mitogen activated protein kinase (MAPK) inhibitor.

In some embodiments, provided herein are methods of reversing endosomal dysfunction in a subject having DLB but no substantial tau pathology, the methods comprising administering to the subject a selective p38α mitogen activated protein kinase (MAPK) inhibitor.

In some embodiments, the MAPK inhibitor is neflamapimod. In some embodiments, neflamapimod is administered at 40 mg TID.

In some embodiments, the subject has cholinergic neurodegeneration in the basal forebrain. In some embodiments administration of neflamapimod results in diminished symptomatic effects of cholinergic neurodegeneration in the basal forebrain of the subject.

In some embodiments, the subject has synaptic dysfunction in the medial septum, neuronal cell loss in the hippocampus, neuronal loss in the medial septum, or neuronal cell loss in the vertical limb of the nucleus of the diagonal band. In some embodiments, the neuronal cell loss in the hippocampus is in CA2-3 regions of the hippocampus.

In some embodiments, the subject has deficits in:
(a) attention, verbal fluency, episodic memory as measured by Attention Composite z-score,
(b) deficits in mobility as measured by Timed Up and Go (TUG),
(c) memory, orientation, judgment and problem solving, community affairs, home and hobbies performance, and personal care as measured by Clinical Dementia Rating Scale (CDR-SB), and/or
(d) deficits in neuropsychological activity as measured by Neuropsychological Test Battery (NTB).

In some embodiments, the subject has alpha synuclein deposits in the hippocampus.

In some embodiments, the subject does not have Alzheimer's Disease.

In some embodiments, neflamapimod is administered to a subject having DLB and a plasma ptau181 level of less than 3 μg/mL.

In some embodiments, neflamapimod is administered to a subject having DLB and a plasma ptau181 level of less than 2.5 pg/mL.

In some embodiments, neflamapimod is administered to a subject having DLB and a plasma ptau181 level of less than 2 μg/mL.

In some embodiments, neflamapimod is administered to a subject having DLB and a plasma ptau181 level of less than 1 μg/mL.

In some embodiments, the subject is also receiving a cholinesterase inhibitor.

In some embodiments the daily amount of neflamapimod administered is equivalent to a dose of 40 mg (TID).

In some embodiments, a subject administered neflamapimod is also receiving a cholinesterase inhibitor therapy. In some embodiments, a subject having a plasma ptau level of less than 2.2 pg/mL and receiving a cholinesterase therapy is administered neflamapimod at a dose of at least 40 mg TID.

### BRIEF DESCRIPTION OF THE DRAWINGS

FIG. 1 shows clinical trial results in subjects receiving neflamapimod for Attention Composite z-score for tests within Neuropsychological Test Battery (NTB) that assess information processing speed. Data are presented as output (Mean, 95% CI) of analysis change from baseline utilizing Mixed Model for Repeated Measures (MMRM). Improvement is reflected as increases in NTB and Attention Composite z-scores.

FIG. 2A shows clinical trial results in subjects receiving neflamapimod for Clinical Dementia Rating Scale (CDR-SB). Data are presented as output (Mean, 95% CI) of analysis change from baseline utilizing Mixed Model for Repeated Measures (MMRM). Improvement is reflected as a decrease in CDR-SB score. 40 mg TID vs. placebo are plotted.

FIG. 2B shows clinical trial results in subjects receiving neflamapimod for Timed Up and Go (TUG) testing. Data are presented as output (Mean, 95% CI) of analysis change from baseline utilizing Mixed Model for Repeated Measures (MMRM). Improvement is reflected as a decrease in time to complete the TUG test. 40 mg TID vs. placebo are plotted.

FIG. 3 shows clinical trial results in subjects receiving neflamapimod for the International Shopping List Test (ISLT). Data are presented as output (Mean, 95% CI) of analysis change from baseline utilizing Mixed Model for Repeated Measures (MMRM). Improvement is reflected by an increase in ISLT score.

### DEFINITIONS

Carrier: The term “carrier” refers to any chemical entity that can be incorporated into a composition containing an active agent (e.g., a p38 MAPKα inhibitor such as neflamapimod) without significantly interfering with the stability and/or activity of the agent (e.g., with a biological activity of the agent). In certain embodiments, the term “carrier” refers to a pharmaceutically acceptable carrier.

Formulation. The term “formulation” as used herein refers to a composition that includes at least one active agent (e.g., p38 MAPKα inhibitor such as neflamapimod) together with one or more carriers, excipients or other pharmaceutical additives for administration to a patient. In general, particular carriers, excipients and/or other pharmaceutical additives are selected in accordance with knowledge in the art to achieve a desired stability, release, distribution and/or activity of active agent(s) and which are appropriate for the particular route of administration.

Pharmaceutically acceptable carrier, adjuvant, or vehicle. The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Therapeutically effective amount and effective amount. As used herein, and unless otherwise specified, the terms “therapeutically effective amount” and “effective amount” of an agent refer to an amount sufficient to provide a therapeutic benefit in the treatment, prevention and/or management of a disease, disorder, or condition, e.g., to delay onset of or minimize (e.g., reduce the incidence and/or magnitude of) one or more symptoms associated with the disease, disorder or condition to be treated. In some embodiments, a composition may be said to contain a “therapeutically effective amount” of an agent if it contains an amount that is effective when administered as a single dose within the context of a therapeutic regimen. In some embodiments, a composition may be said to contain a “therapeutically effective amount” of an agent if it contains an amount that is effective when administered as more than one dose (e.g., two doses, three doses, or four or more doses) within the context of a therapeutic regimen. In some embodiments, a therapeutically effective amount is an amount that, when administered as part of a dosing regimen, is statistically likely to delay onset of or minimize (reduce the incidence and/or magnitude of) one or more symptoms or side effects of a disease, disorder or condition.

Treat or Treating. The terms “treat” or “treating,” as used herein, refer to partially or completely alleviating, inhibiting, delaying onset of, reducing the incidence of, yielding prophylaxis of, ameliorating and/or relieving or reversing a disorder, disease, or condition, or one or more symptoms or manifestations of the disorder, disease or condition.

Unit Dose. The expression “unit dose” as used herein refers to a physically discrete unit of a formulation appropriate for a subject to be treated (e.g., for a single dose); each unit containing a predetermined quantity of an active agent selected to produce a desired therapeutic effect when administered according to a therapeutic regimen (it being understood that multiple doses may be required to achieve a desired or optimum effect), optionally together with a pharmaceutically acceptable carrier, which may be provided in a predetermined amount. The unit dose may be, for example, a volume of liquid (e.g., an acceptable carrier) containing a predetermined quantity of one or more therapeutic agents, a predetermined amount of one or more therapeutic agents in solid form (e.g., a tablet or capsule), a sustained release formulation or drug delivery device containing a predetermined amount of one or more therapeutic agents, etc. It will be appreciated that a unit dose may contain a variety of components in addition to the therapeutic agent(s). For example, acceptable carriers (e.g., pharmaceutically acceptable carriers), diluents, stabilizers, buffers, preservatives, etc., may be included as described infra. It will be understood, however, that the total daily usage of a formulation of the present invention will be decided by the attending physician within the scope of sound medical judgment. The specific effective dose level for any particular subject may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts. In some embodiments, a unit dose of a p38 MAPKα inhibitor, such as neflamapimod is about 1 mg, 3 mg, 5 mg, 10 mg, 15 mg, 20 mg, 25 mg, 30 mg, 35 mg, 40 mg, 45 mg, 50 mg, 100 mg, 125 mg, or 250 mg.

### DETAILED DESCRIPTION OF CERTAIN EMBODIMENTS

The present invention provides, among other things, compositions and methods for treating subjects that have Dementia with Lewy Bodies (DLB) and associated pathology, but no substantial tau pathology, by administering a composition comprising a selective p38 MAPKα inhibitor, such as neflamapimod. Tau pathology may be characterized by measurement of circulating (e.g., blood or plasma) levels of phospho-tau (ptau).

In some embodiments, a plasma ptau181 level of less than 2.2 pg/mL is indicative of no substantial tau pathology, and a plasma ptau181 level of equal to or greater than 2.2 pg/mL is indicative of the presence of tau pathology in brain.

In some embodiments, tau pathology in a subject is characterized by level of ptau217 in plasma, positron emission topography (PET) in brain, and/or level of amyloid beta (Aβ) 42 in cerebrospinal fluid.

Accordingly, in some embodiments, provided herein is a method of treating DLB in a subject that does not have substantial tau pathology (or Alzheimer's Disease-like pathology). In some embodiments, ptau that is measured to determine tau pathology is ptau181. In some embodiments, ptau that is measured to determine tau pathology is ptau217. In some embodiments, ptau that is measured to determine tau pathology is ptau231. See e.g., Bayoumi et al., Alzheimer's Research & Therapy, 13:198 (2021) for exemplary ptau assays and comparative results.

Methods of identifying or diagnosing AD-like pathology are known. For example, AD pathology can be determined by measuring a level of beta-amyloid, precursor, or fragment thereof in a subject's blood, plasma, CSF, or brain.

In some embodiments, plasma ptau181 is used as a marker of tau pathology and the threshold value for determining whether a subject has tau pathology is 2.2 pg/mL. In some embodiments, the threshold value for ptau181 is at least 1.5 pg/mL. In some embodiments, the threshold value for ptau181 is at least 1 pg/mL, 1.6 pg/mL, 1.7 pg/mL, 1.8 pg/mL, 1.9 pg/mL, 2 pg/mL, 2.1 pg/mL, 2.2 pg/mL, 2.3 pg/mL, 2.4 pg/mL, 2.5 pg/mL, 2.6 pg/mL, 2.7 pg/mL, 2.8 pg/mL, 2.9 pg/mL, or 3 pg/mL or more.

In some embodiments, the invention provides compositions and methods for treating subjects susceptible or at risk of development or progression of DLB.

Various aspects of the invention are described in detail in the following sections. The use of sections is not meant to limit the invention. Each section can apply to any aspect of the invention. In this application, the use of “or” means “and/or” unless stated otherwise.

### Dementia With Lewy Bodies

There is currently no therapy available for DLB that reverses and/or slows disease progression. A therapeutic intervention such as neflamapimod, that targets synaptic dysfunction, has the potential to both reverse existing synaptic deficits and slow further decline.

The central feature of DLB is a progressive dementia, i.e. decline in cognition associated with functional deficits, that is characterized by deficits in attention and executive function but can include memory deficits. In addition, associated symptoms include fluctuation in attentiveness, slowness of movement, rigidity, REM sleep disruption, visual hallucinations, anosmia, fluctuation in attentiveness, depression, apathy, and autonomic nervous system dysregulation. DLB is associate with deposits of alpha-synuclein in cells, known as Lewy bodies or Lewy neurites.

The medial septum (also known as Ch1) and the vertical limb of the diagonal band (also known as Ch2) provide cholinergic innervation to the hippocampus. Loss of neurons in the medial septum nucleus and vertical limb of the diagonal band is a specific feature of DLB that differentiates it from AD (Fujishiro et al., Acta Neuropathol, 111:109-1114 (2006). It has been discovered herein that loss of cholinergic neurons in the medial septum can be inhibited by administration of a selective p38 α MAPK inhibitor, neflamapimod.

### Neflamapimod

Many extracellular stimuli, including pro-inflammatory cytokines and other inflammatory mediators, elicit specific cellular responses through the activation of mitogen-activated protein kinase (MAPK) signaling pathways. MAPKs are proline-targeted serine-threonine kinases that transduce environmental stimuli to the nucleus. Once activated, MAPKs activate other kinases or nuclear proteins through phosphorylation, including potential transcription factors and substrates. The four isoforms (α, β, δ, and γ) of p38 MAP kinase comprise one specific family of MAPKs that mediate responses to cellular stresses and inflammatory signals. Neflamapimod is a selective small-molecule inhibitor of the alpha isoform of p38 MAPK. Neflamapimod, also known as VX-745, has a chemical name of 5-(2,6-dichlorophenyl)-2-(2,4-difluorophenylthio)-6H-pyrimido [1,6-b]pyridazin-6-one

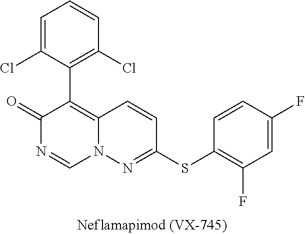 [SMILES: O=c1ncn2nc(Sc3ccc(F)cc3F)ccc2c1-c1c(Cl)cccc1Cl]

### Pharmaceutical Compositions

In some embodiments, a provided method comprises administering to a patient a pharmaceutical composition comprising neflamapimod together with one or more therapeutic agents and a pharmaceutically acceptable carrier, adjuvant, or vehicle. In some embodiments, a pharmaceutical composition is provided comprising a dose of neflamapimod together with one or more therapeutic agents and a pharmaceutically acceptable carrier, adjuvant, or vehicle, wherein the dose of neflamapimod results in an average blood concentration of from about 1 ng/mL to about 15 ng/ml, from about 1 ng/ml to about 10 ng/mL, from about 5 ng/mL to about 15 ng/ml, or from about 5 ng/ml to about 10 ng/ml. In some embodiments, the dose of neflamapimod results in an average blood concentration of 8 ng/ml. Table 2 of WO 2017/185073 illustrates neflamapimod plasma concentration values by post-dose collection time interval, and is incorporated by reference herein.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Dosing

In some embodiments, a composition is administered in a therapeutically effective amount and/or according to a dosing regimen that is correlated with a particular desired outcome (e.g., with treating or reducing risk for disease).

In some embodiments, provided compositions are administered in a therapeutically effective amount and/or according to a dosing regimen that is correlated with a particular desired outcome (e.g., reduction in symptoms, etc.).

Alternatively or additionally, in some embodiments, an appropriate dose or amount is determined through use of one or more in vitro or in vivo assays to help identify desirable or optimal dosage ranges or amounts to be administered.

In various embodiments, provided compositions are administered at a therapeutically effective amount. Generally, a therapeutically effective amount is sufficient to achieve a meaningful benefit to the subject (e.g., treating, modulating, curing, preventing and/or ameliorating the underlying disease or condition). In some embodiments, methods of treating a subject having DLB comprise administering a therapeutically effective amount of a selective p38α inhibitor. In some embodiments, methods of treating a subject having DLB comprise administering a therapeutically effective amount of neflamapimod.

In some embodiments, a composition is provided as a pharmaceutical formulation. In some embodiments, a pharmaceutical formulation is or comprises a unit dose amount for administration in accordance with a dosing regimen correlated with achievement of disease reduction in symptoms of DLB, arrest or decrease in rate of decline of function due to DLB.

In some embodiments, a formulation comprising provided compositions as described herein is administered as a single dose. In some embodiments, a formulation comprising provided compositions as described herein is administered as two doses. In some embodiments, a formulation comprising provided compositions as described herein is administered at regular intervals. Administration at an “interval,” as used herein, indicates that the therapeutically effective amount is administered periodically (as distinguished from a one-time dose). The interval can be determined by standard clinical techniques. In some embodiments, a formulation comprising provided compositions as described herein is administered twice weekly, thrice weekly, every other day, daily, twice daily, or every eight hours.

In some embodiments, a formulation comprising provided compositions as described herein is administered once daily. In some embodiments, a formulation comprising provided compositions as described herein is administered twice daily. In some embodiments, the twice daily administering occurs from about 9 to 15 hours apart. In some embodiments the twice daily administering occurs about 12 hours apart. In some embodiments, a formulation comprising from about 40 mg to about 250 mg of neflamapimod is administered twice daily. In some embodiments, a formulation comprising compositions as described herein is administered three times daily. In some embodiments, the administering occurs when the patient is in a fed state. In some embodiments, the administering occurs within 30 to 60 minutes after the subject has consumed food. In some embodiments, the administering occurs when the patient is in a fasted state. The administration interval for a single individual need not be a fixed interval, but can be varied over time, depending on the needs of the individual.

In some embodiments, a formulation comprising provided compositions as described herein is administered at regular intervals. In some embodiments, a formulation comprising provided compositions as described herein is administered at regular intervals for a defined period. In some embodiments, a formulation comprising provided compositions as described herein is administered at regular intervals for 2 years, 1 year, 11 months, 10 months, 9 months, 8 months, 7 months, 6 months, 5 months, 4 months, 3 months, 2 months, a month, 3 weeks, 2, weeks, a week, 6 days, 5 days, 4 days, 3 days, 2 days or a day. In some embodiments, a formulation comprising provided compositions as described herein is administered at regular intervals for 16 weeks.

### EXEMPLIFICATION

The following examples are provided for illustrative purposes and are not intended to limit the scope of the invention.

### Example 1

This example demonstrates that neflamapimod is particularly efficacious for treatment of subjects that have DLB without substantial tau pathology.

Treatment effects of neflamapimod in a mid-to-moderate DLB patient population receiving cholinesterase inhibitor therapy was evaluated in a 91-patient, 16-week placebo-controlled phase 2 study (“AscenD-LB Study”) in mild-to-moderate DLB, neflamapimod demonstrated significant improvement, relative to placebo, in cognition (assessed by DLB specific Neuropsychological Test Battery (NTB), motor function (assessed by the Timed-Up-and-Go (TUG) Test), and cognition and function (assessed by Clinical Dementia Rating Scale sum-of-boxes (CDR-SB)).

Up to half of patients with DLB have tau pathology, or “AD copathology” (van der Lee et al, 2021) and such co-pathology may impact response to cholinesterase inhibitors (Graff-Radford et al, 2012). In patients with DLB, plasma phospho-tau (either ptau217 or ptau181) correlates with tau-PET signal in the temporal cortex and predicts abnormal tau-PET status and CSF β-amyloid status (Hall et al, 2021). Accordingly, plasma ptau181 levels were assessed in plasma samples obtained during the screening phase of AscenD-LB. The results on the association of tau pathology to treatment outcome in AscenD-LB are described below.

### Dosing Regimen

With the objective of uniformly achieving a target average plasma drug concentration of 20 nM patients, dosing regimen after randomization to neflamapimod or placebo was based on weight:
Weight<80 kg: 40 mg neflamapimod capsule or matching placebo capsule BID
Weight≥80 kg: 40 mg neflamapimod capsule or matching placebo capsule TID

However, in the study, 40 mg TID achieved target plasma drug concentration; but 40 mg BID did not, missing by approximately 30-40%.

Trough plasma drug concentrations 50% lower with 40 mg BID vs. 40 mg TID

As a result, efficacy analyses compared: (1) all neflamapimod (i.e., including 40 mg BID and 40 mg TID) vs. placebo; (2) 40 mg TID (dose group that achieved target concentration) vs. placebo; and (3) 40 mg TID vs. placebo TID.

To evaluate treatment effects, linear mixed effects model for repeated measures (LMMRM) was utilized to compare outcomes in NFMD40 mg TID, the dose group that achieved therapeutic plasma drug levels and demonstrated efficacy in the main analyses reported previously, to (1) all placebo recipients, and (2) the matched higher weight placebo-recipients (placebo TID). Plasma ptau181 levels were determined by SIMOA pTau181 Assay (Quanterix) at the VU Medical Center, where the in-house defined cut-off for tau pathology was set at 2.2 pg/mL.

### Baseline Disease Characteristics

At baseline, 22 of 41 (53%) of placebo and 22 of 42 (54%) of neflamapimod participants in the efficacy analysis population (baseline and on-treatment data on at least one efficacy endpoint) had plasma ptau181 <2.2 pg/mL (i.e., those predicted to not have tau pathology). See Table 1.

TABLE 1
Baseline characteristics by plasma ptau181 status

| | Baseline plasma ptau181 < 2.2 pg/mL | | | | | | Baseline plasma ptau181 ≥ 2.2 pg/mL | | | | | |
|---|---|---|---|---|---|---|---|---|---|---|---|---|
| | Placebo | | NFMD | | All | | Placebo | | NFMD | | All | |
| | (N = 23) | | (N = 22) | | (N = 45) | | (N = 20) | | (N = 20) | | (N = 40) | |
| Age (yrs) | 70.7 | (6.0) | 72.6 | (6.8) | 71.7 | (6.4) | 73.8 | (7.5) | 74.1 | (6.7) | 73.9 | (7.0) |
| Male | 87% | | 82% | | 84% | | 85% | | 85% | | 85% | |
| CDR Sum of | 4.3 | (2.0) | 4.7 | (2.1) | 4.5 | (2.1) | 6.1 | (3.4) | 5.1 | (2.1) | 5.5 | (2.8) |
| Boxes | | | | | | | | | | | | |
| MMSE | 24 | (3.8) | 24 | (3.0) | 24 | (3.4) | 23 | (3.6) | 22 | (3.7) | 22 | (3.6) |
| ISLT | 15 | (6.0) | 15 | (6.2) | 15 | (6.0) | 13 | (4.6) | 12 | (5.5) | 13 | (5.0) |
| (Immediate) | | | | | | | | | | | | |
| ISLT (Delayed) | 5 | (2.3) | 4 | (2.8) | 4 | (2.5) | 4 | (1.8) | 4 | (2.8) | 4 | (2.4) |
| ISLT | 10.7 | (1.3) | 10.1 | (1.6) | 10.4 | (1.4) | 9.5 | (2.0) | 10.8 | (1.2) | 10.1 | (1.8) |
| (Recognition) | | | | | | | | | | | | |
| Timed Up and | 14 | (7.7) | 12 | (3.8) | 13 | (6.1) | 13 | (3.6) | 14 | (4.5) | 13 | (4.1) |

| | | | | | | |
|---|---|---|---|---|---|---|
| Go (seconds) | | | | | | |
| Fluctuating | 56% | 54% | 53% | 60% | 70% | 65% |
| cognition | | | | | | |
| Visual | 65% | 50% | 58% | 45% | 70% | 58% |
| hallucinations | | | | | | |
| REM sleep | 83% | 59% | 71% | 65% | 55% | 60% |
| disorder | | | | | | |
| Parkinsonism | 87% | 73% | 80% | 85% | 80% | 83% |
| ≥2 core clinical | 91% | 73% | 82% | 85% | 90% | 88% |
| features* | | | | | | |

*Fluctuating cognition, visual hallucinations, REM sleep disorder, or parkinsonism Mean (SD), except when shown as percentage
Note:
For ISLT (delayed) and ISLT (recognition), missing data in 1 NFMD participant with baseline ptau < 2.2 pg/mL and 1 placebo participant with ptau > 2.2 pg/mL. Also missing TUG data in 1 participant with baseline ptau181 ≥ 2.2 pg/mL.

### Results

For all four endpoints that had shown significant treatment effects in the main analysis (NTB z-sore, attention z-score, TUG, CDR-SB), descriptive evaluation of the results by baseline ptau181 status revealed that positive treatment effects were to the patients who were below the threshold (2.2 pg/mL) for having tan pathology, with no discernible treatment effect (positive or negative) with baseline plasma ptau181 levels above the threshold, i.e., those with tau pathology.

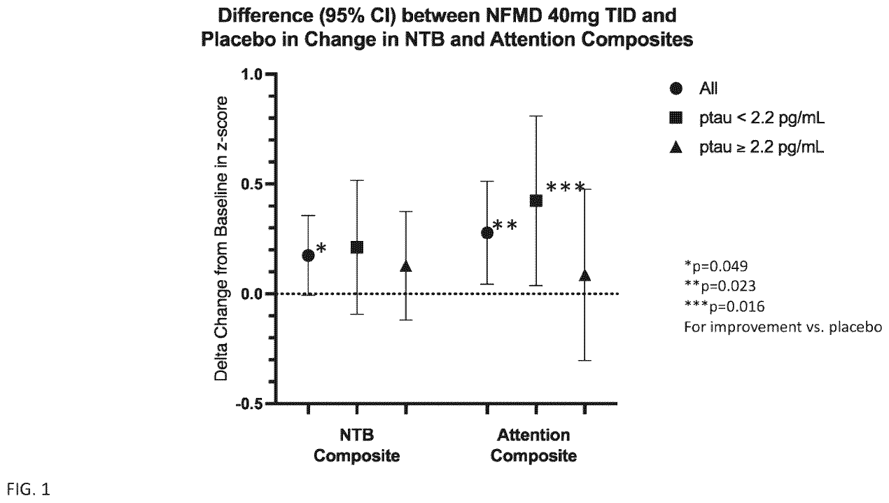

When LMMRM analysis was confined to patients who had ptau181 <2.2 pg/mL at baseline, for both comparisons significant positive treatment favoring NFMD 40 mg TID were seen for the attention composite z-score (p=0.021 vs. placebo, difference=0.42 95% CI: 0.07, 0.78; p=0.034 vs. placebo TID, difference=0.46 z-score, 95% CI: 0.04, 0.88). Sec FIG. 1.

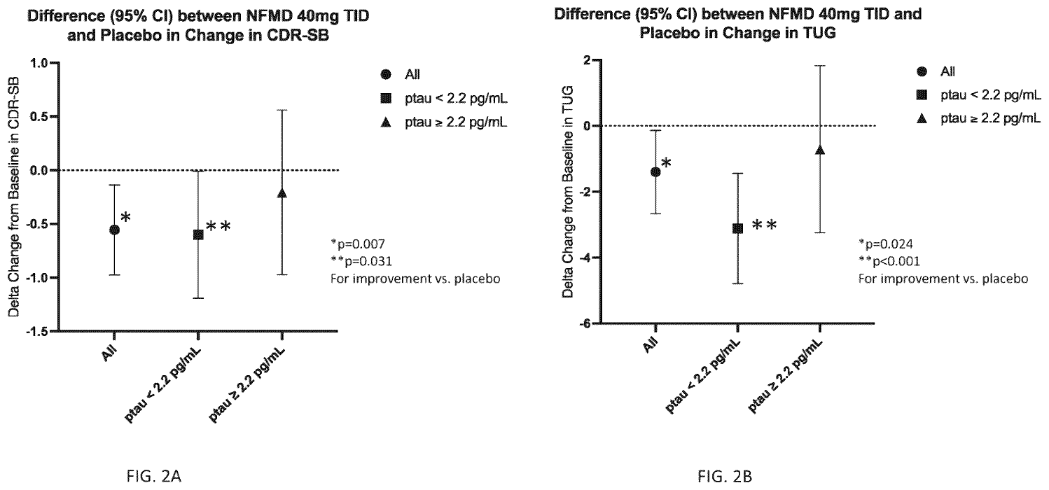

When LMMRM analysis was confined to patients who had ptau181 <2.2 pg/mL at baseline, for both comparisons significant positive treatment favoring NFMD 40 mg TID were seen for the TUG (p<0.001 vs. placebo, difference=−3.1, 95% CI: −4.7, −1.6; p=0.010 vs. placebo TID, difference=−3.5 seconds, 95% CI: −6.1, −0.9). See FIG. 2B.

When LMMRM analysis was confined to patients who had ptau181 <2.2 pg/mL at baseline, for both comparisons significant positive treatment favoring NFMD 40 mg TID were seen for the CDR-SB (p=0.031 vs. placebo, difference=−0.60, 95% CI: −1.14, −0.06; p=0.009 vs. placebo TID, difference=−0.93 points, 95% CI: −1.61, −0.25). See FIG. 2A.

When LMMRM analysis was confined to patients who had ptau181 <2.2 pg/mL at baseline, for both comparisons significant positive treatment favoring NFMD 40 mg TID were seen for the NTB z-score, though numerically favoring NFMD 40 mg TID treatment (difference=0.21 vs. placebo, 95% CI: −0.07, 0.49; difference=0.25 vs. placebo TID, 95% CI: −0.07, 0.57), with the limited sample size the differences were not significant (p=0.13 vs. placebo, p=0.12 vs. placebo TID). However, for the NTB, a significant PK-PD relationship (p=0.035, r=0.46, for estimated trough plasma neflamapimod drug concentration vs. NTB z-score score) was evident.

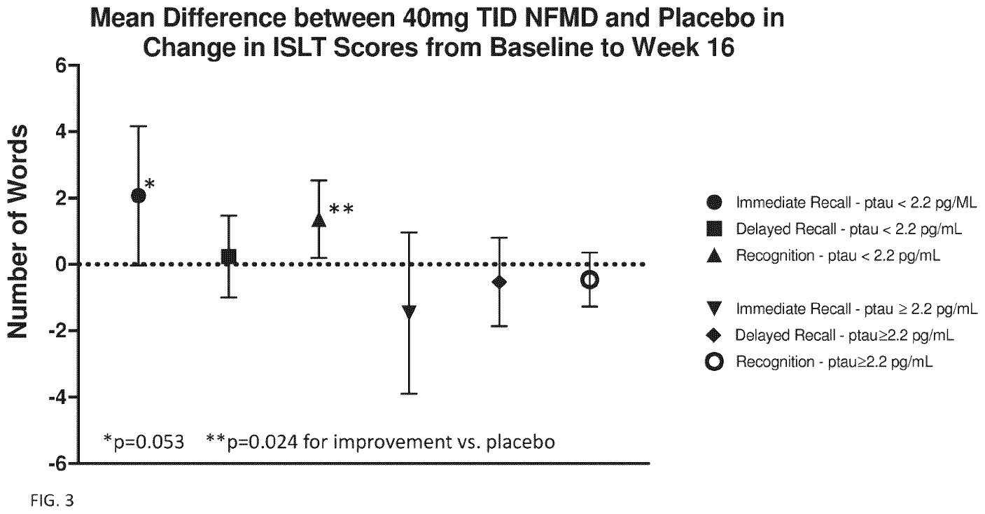

When LMMRM analysis was confined to patients who had ptau181 <2.2 pg/mL at baseline, for both comparisons, significant positive trends favoring NFMD 40 mg TID were seen for ISLD immediate recall (p=0.053 vs. placebo, difference=2.1 words 95% CI: −0.0, 4.2; p=0.063 vs. placebo TID, difference=2.3 words, 95% CI: −0.1, 4.7) and significant positive improvement favoring NFMD 40 mg TID for ISLD recognition (p=0.024 vs. placebo, difference=1.4 words, 95% CI: 0.2, 2.5; p=0.035 vs. placebo TID, difference=0.9 words, 95% CI: 0.1, 1.7). See FIG. 3. The positive effect on ISLD recognition is consistent with an effect on working memory.

Efficacy analyses of the major endpoints show that the response in the patients with baseline plasma ptau181 <2.2 pg/mL (i.e., those without substantial tau pathology) appears to be better than the response in patients with baseline plasma ptau181 levels ≥2.2 pg/mL (i.e., those with tau pathology in brain, and potentially mixed AD-related pathology).

When the protocol-specified efficacy analysis (mixed model for repeated measures) is confined to patients without substantial tau pathology, the magnitude of the neflamapimod treatment effect relative to placebo is substantial and clinically important. See Table 2.

TABLE 2
Summary of neflamapimod efficacy in DLB patient populations
without tau pathology (baseline plasma ptau181 < 2.2 pg/mL)

| | Improvement in all | | Improvement in | | Improvement in | |
|---|---|---|---|---|---|---|
| | neflamapimod vs. | | neflamapimod 40 mg | | neflamapimod 40 mg | |
| | placebo | | TID vs. placebo | | TID vs. placebo TID | |
| | | Effect | | Effect | | Effect |
| Cognition: | p-value | size | p-value | size | p-value | Size |
| NTB* | >0.2 | 0.15 | 0.133 | 0.56 | 0.123 | 0.61 |
| Attention | 0.185 | 0.29 | 0.021 | 0.78 | 0.034 | 0.70 |
| Timed up and | 0.024 | 0.40 | <0.001 | 0.74 | 0.010 | 0.70 |
| Go | | | | | | |
| CDR-SB | 0.129 | 0.58 | 0.031 | 0.70 | 0.009 | 0.98 |

Compared to the results in the overall population, the magnitude of the neflamapimod treatment effect relative to placebo for the individual endpoints was 1.5 to 2.0-fold greater in the population with plasma ptau181 <2.2 pg/mL at baseline.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

What is claimed is:

1. A method for treating a subject having Dementia with Lewy Bodies (DLB) but no substantial tau pathology, the method comprising administering a selective p38 mitogen activated protein kinase (MAPK) inhibitor to the subject.

2. The method of claim 1, wherein the p38α mitogen activated protein kinase (MAPK) inhibitor is neflamapimod.

3. The method of claim 1, wherein no substantial tau pathology in a subject is characterized by level of ptau181 in plasma.

4. The method of claim 1, wherein no substantial tau pathology in a subject is characterized by level of ptau217 in plasma.

5. The method of claim 1, wherein no substantial tau pathology in a subject is characterized by positron emission topography (PET) of brain.

6. The method of claim 1, wherein no substantial tau pathology in a subject is associated with a level of amyloid beta (Aβ) 42 in cerebrospinal fluid in the subject that is lower than that of subjects diagnosed as having Alzheimer's Disease.

7. The method of claim 5, wherein no substantial tau pathology in a subject is characterized by a level of plasma ptau lower than that of a subject having Alzheimer's Disease or Alzheimer's Disease related pathology as measured in a Simoa ptau181 assay.

8. A method of inhibiting neuronal loss in the central nervous system in a subject having DLB but no substantial tau pathology, the method comprising administering to the subject a selective p38α mitogen activated protein kinase (MAPK) inhibitor.

9. A method of reversing endosomal dysfunction in a subject having DLB but no substantial tau pathology, the method comprising administering to the subject a selective p38α mitogen activated protein kinase (MAPK) inhibitor.

10. The method of claim 9, wherein the MAPK inhibitor is neflamapimod.

11. The method of claim 10, wherein the neflamapimod is administered at 40 mg TID.

12. The method of claim 1, wherein the subject has cholinergic neurodegeneration in the basal forebrain.

13. The method of claim 12, wherein the administration results in diminished symptomatic effects of cholinergic neurodegeneration in the basal forebrain of the subject.

14. The method of claim 1, wherein the subject has synaptic dysfunction in the medial septum, neuronal cell loss in the hippocampus, neuronal loss in the medial septum, or neuronal cell loss in the vertical limb of the nucleus of the diagonal band.

15. The method of claim 14, wherein the neuronal cell loss in the hippocampus is in CA2-3 regions of the hippocampus.

16. The method of claim 1, wherein the subject has deficits in:
(a) attention, verbal fluency, episodic memory as measured by Attention Composite z-score,
(b) deficits in mobility as measured by Timed Up and Go (TUG),
(c) memory, orientation, judgment and problem solving, community affairs, home and hobbies performance, and personal care as measured by Clinical Dementia Rating Scale (CDR-SB), and/or
(d) deficits in neuropsychological activity as measured by Neuropsychological Test Battery (NTB).

17. The method of claim 1, wherein the subject has alpha synuclein deposits in the hippocampus.

18. The method of claim 1, wherein the subject does not have Alzheimer's Disease.
